(19) United States
(12) Patent Application Publication
(10) Pub. No.: US 2022/0354817 A1
(43) Pub. Date: Nov. 10, 2022

(54) PHARMACEUTICAL COMPOSITION FOR PREVENTING OR TREATING SARCOPENIA CONTAINING NON-NATURAL AMINO ACID DERIVATIVE

(71) Applicant: MYO-TEC-SCI, Seoul (KR)

(72) Inventors: Hyeon Soo KIM, Seoul (KR); Jae Ho SHIM, Seoul (KR)

(21) Appl. No.: 17/763,463

(22) PCT Filed: Sep. 24, 2020
(86) PCT No.: PCT/KR2020/012994

§ 371 Date: Mar. 24, 2022

(30) Foreign Application Priority Data
Sep. 24, 2019 (KR) 10-2019-0117223
Sep. 23, 2020 (KR) 10-2020-0123047

(51) Int. Cl. A61K 31/223; A61P 21/06

(52) U.S. Cl. CPC A61K 31/223; A61P 21/06

(57) ABSTRACT

According to an embodiment of the present invention, provided is a composition for preventing or treating sarcopenia containing a non-natural amino acid derivative as an active ingredient.

### CROSS-REFERENCE TO RELATED APPLICATIONS

This application is a U.S. national stage entry of International Application No. PCT/KR2020/012994, filed Sep. 24, 2020, which claims priority to Korean Application No. 10-2019-0117223, filed Sep. 24, 2019, and Korean Application No. 10-2020-0123047, filed Sep. 23, 2020, the entire disclosures of which are incorporated herein by

### REFERENCE

### Technical Field

The present disclosure relates to a pharmaceutical composition for preventing or treating sarcopenia containing a non-natural amino acid derivative as an active ingredient.

### BACKGROUND

The concept of sarcopenia started when Irwin Rosenberg introduced the term “sarcopenia” in 1989. Upon reviewing its origin in Greek, it is a compound word of “sarco” meaning muscle and “penia” meaning reduced. Sarcopenia refers to a decrease in muscle strength due to a decrease in muscle mass associated with aging. Here, “muscle” refers to skeletal muscle and is unrelated to smooth muscle. In other words, sarcopenia refers to the loss of skeletal muscle mass mainly distributed in the extremities, and is distinguished from cachexia, which is a state of significant muscle loss in the late stages of a malignant tumor, muscle wasting due to an acute disease such as influenza, or a disease of the muscle itself (primary muscle disease).

Recently, the prevalence of osteoporosis and sarcopenia is also increasing rapidly as the age group of the elderly 65 years or older has rapidly increased. It is estimated that the gradual decrease in muscle mass occurs after the age of 40 and decreases by 8% every 10 years until the age of 70. It is known that thereafter, even more rapid decreases occur, which can occur by as much as 15% every 10 years. Many follow-up studies have shown that the physiological changes that occur in the elderly are diverse, and in general, muscle mass and bone density decrease simultaneously with increasing age.

There are three major treatment methods for sarcopenia. The first is exercise. It has been reported that exercise increases the protein synthesis ability of skeletal muscle in the short term, and increases muscle strength or motility of the elderly. However, it is not suitable for long-term treatment. Second, testosterone or anabolic steroid can be used as drug treatment, but this induces masculinization in women, and in men, it exhibits side effects such as prostate symptoms. Other approved prescriptions include DHEA (dehydroepiandrosterone) and growth hormone, and studies have reported that it can be used as a treatment method for sites that contain SARMs (selective androgen receptor modulators). In addition, although diet is known as a treatment method, nutritional evaluation shows that malnutrition or modern eating habits are inadequate to maintain adequate total body mass.

Myostatin is a polypeptide growth factor belonging to the superfamily of TGF-β. TGF-β has a large amount of isoforms, which are known to be involved in cell proliferation, apoptosis, differentiation, and bone formation and maintenance (Massague & Chen, 2000). Myostatin belongs to growth differentiation factor (GDF) number 8 among them, is involved in tissue growth and development, and works by activating the Smad signaling system. In addition, it has been reported that the p21 gene inhibits cell cycle and progenitor cell proliferation, thereby affecting bone formation and regeneration. It is known that myostatin is mainly produced in skeletal muscle cells and causes muscle loss and muscle strength reduction in an autocrine manner, and that by inhibiting the expression of IGF-1 or Follistatin involved in muscle hypertrophy, protein synthesis and cell proliferation in myoblast are inhibited.

### SUMMARY

Under this background, the present inventors made intensive efforts to discover a substance capable of treating sarcopenia by inhibiting myostatin expression and promoter activity, which causes muscle loss and muscle strength reduction, and as a result, found that it could be used for preventing or treating sarcopenia by inhibiting the increase in mRNA expression and the production of non-natural amino acid derivative myostatin protein, and then completed the present disclosure.

It is an aspect of the present disclosure to provide a pharmaceutical composition for preventing or treating sarcopenia containing a non-natural amino acid derivative as an active ingredient.

However, the aspects of the present disclosure are not limited to those mentioned above, and other aspects not mentioned herein will be clearly understood by those skilled in the art from the following description.

### Technical Solutions

According to an example embodiment of the present disclosure, there is provided a pharmaceutical composition for preventing or treating sarcopenia containing, as an active ingredient, a non-natural amino acid derivative consisting of at least one selected from the group consisting of following Formulas 1 to 19 or a mixture of X₂ and at least one selected from the group consisting of following Formulas 1 to 19:

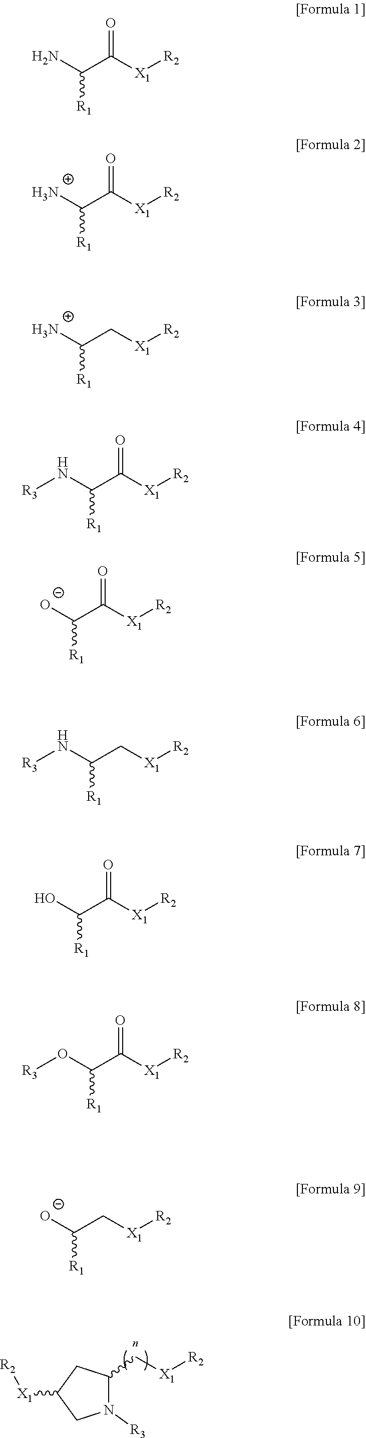

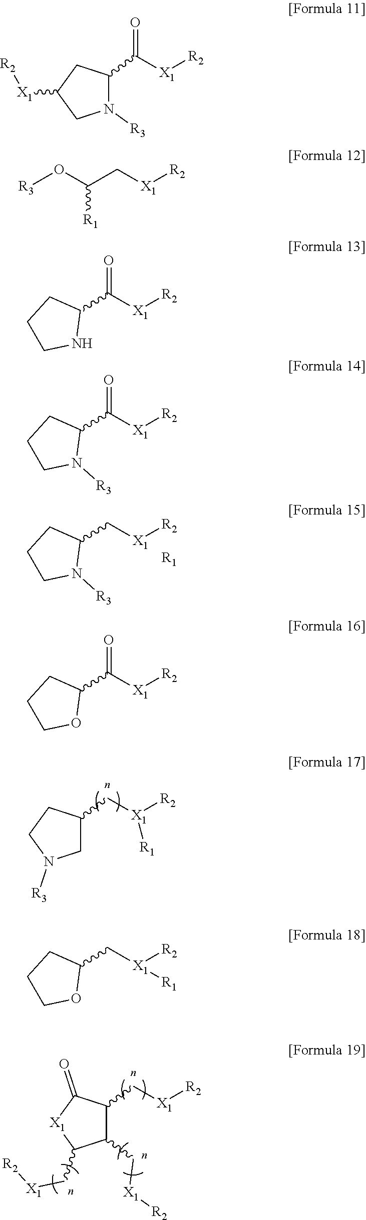

in which,

R₁ is hydrogen, a C1-C10 carboxylic acid, a C1-C10 alkyl group, C1-C10 amine, C1-C10 alcohol, guanidine, phenyl, phenol, indole, or imidazole;

R₂ is hydrogen, a C1-C10 alkyl group, a C1-C10 aryl alkyl group, or D-glucose;

R₃ is t-butyl carbamate, benzyl carbamate, 9-fluorenylmethyl carbamate, an amine group, an ester group or acetamide;

X₁ is N, O, P, S, OH or NH₂;

X₂ is Cl⁻, Br⁻, I⁻, p-toluenesulfonate, CF₃COOH, Na⁺, Mg²⁺, Ca²⁺, H₂O or HCl; and

n is 0, 1 or 2.

According to an aspect, the pharmaceutical composition may inhibit myostatin mRNA or protein expression.

According to an aspect, the pharmaceutical composition may reduce a myostatin promoter activity.

According to an aspect, the non-natural amino acid derivative may be at least one selected from the group consisting of D-Leu-OtBu-HCl, D-Leu-OMe-HCl and D-Leu-BOC—H2O.

According to an aspect, the pharmaceutical composition for preventing or treating sarcopenia may contain 0.001 mM to 10 mM of the non-natural amino acid.

According to an example embodiment of the present disclosure, there is provided a preparation for preventing or treating sarcopenia including a pharmaceutical composition containing, as an active ingredient, a non-natural amino acid derivative consisting of at least one selected from the group consisting of the above Formulas 1 to 19 or a mixture of X₂ and at least one selected from the group consisting of the above Formulas 1 to 19, and at least one selected from the group consisting of a pharmaceutically acceptable carrier, an excipient, a diluent, a stabilizer and a preservative.

According to an aspect, the preparation may have a formulation of powder, granule, tablet, capsule or injection.

The pharmaceutical composition of the present disclosure for preventing or treating sarcopenia containing a non-natural amino acid derivative as an active ingredient inhibits the increase in myostatin protein production and mRNA expression, which directly affects muscle loss and muscle strength reduction, and thus can exhibit a more fundamental preventive or therapeutic effect of sarcopenia.

It should be understood that the effects of the present disclosure are not particularly limited to those described above, and the present disclosure includes all effects that can be deduced from the detailed description of the invention or the configurations of the invention described in the claims.

### BRIEF DESCRIPTION OF DRAWINGS

FIG. 1 illustrates a comparison of myostatin promoter activities of mouse skeletal muscle cells C2C12 in a complete medium containing L-leucine and a medium without L-leucine.

FIG. 2 illustrates a comparison of a myostatin promoter activity of mouse skeletal muscle cells C2C12 after treatment with L-Leu-OtBu-HCl in a medium without L-leucine.

FIG. 3 illustrates a measurement of myostatin mRNA expression levels of mouse skeletal muscle cells C2C12 in a complete medium containing L-leucine and a medium without L-leucine.

FIG. 4 illustrates a comparison of myostatin mRNA expression levels of mouse skeletal muscle cells C2C12 after treatment with L-Leu-OtBu-HCl in a medium without L-leucine.

FIG. 5 illustrates an observation of myostatin promoter activities of mouse skeletal muscle cells C2C12 in a medium without L-leucine (Control) and a medium treated with L-leucine at concentrations of 0.1 mM and 1 mM, and a medium treated with D-Leu-OMe-HCl at concentrations of 0.1 mM and 1 mM.

FIG. 6 illustrates a mutual comparison of myostatin promoter activities of mouse skeletal muscle cells C2C12 in a medium without L-leucine (Control) and a medium treated with L-leucine at concentrations of 0.1 mM and 1 mM, and a medium treated with D-Leu-OMe-HCl at concentrations of 0.1 mM and 1 mM.

FIG. 7 illustrates an observation of myostatin promoter activities of mouse skeletal muscle cells C2C12 in a medium without L-leucine (Control) and a medium treated with L-leucine at concentrations of 0.1 mM and 1 mM, and a medium treated with D-Leu-BOC-H2O at concentrations of 0.1 mM and 1 mM.

FIG. 8 illustrates a mutual comparison of myostatin promoter activities of mouse skeletal muscle cells C2C12 in a medium without L-leucine (Control) and a medium treated with L-leucine at concentrations of 0.1 mM and 1 mM, and a medium treated with D-Leu-BOC—H2O at concentrations of 0.1 mM and 1 mM.

FIG. 9 illustrates a measurement of myostatin mRNA expression levels of mouse skeletal muscle cells C2C12 in a medium without L-leucine (Control) and a medium treated with L-leucine at concentrations of 0.1 mM and 1 mM, and a medium treated with D-Leu-OMe-HCl at concentrations of 0.1 mM and 1 mM.

FIG. 10 illustrates a mutual comparison of myostatin mRNA expression levels of mouse skeletal muscle cells C2C12 in a medium without L-leucine (Control) and a medium treated with L-leucine at concentrations of 0.1 mM and 1 mM, and a medium treated with D-Leu-OMe-HCl at concentrations of 0.1 mM and 1 mM.

FIG. 11 illustrates a measurement of myostatin mRNA expression levels of mouse skeletal muscle cells C2C12 in a medium without L-leucine (Control) and a medium treated with L-leucine at concentrations of 0.1 mM and 1 mM, and a medium treated with D-Leu-BOC-H2O at concentrations of 0.1 mM and 1 mM.

FIG. 12 illustrates a mutual comparison of myostatin mRNA expression levels of mouse skeletal muscle cells C2C12 in a medium without L-leucine (Control) and a medium treated with L-leucine at concentrations of 0.1 mM and 1 mM, and a medium treated with D-Leu-BOC—H2O at concentrations of 0.1 mM and 1 mM.

FIG. 13 illustrates an observation of differentiation degrees of mouse skeletal muscle cells on days 1, 6, and 9 after giving media containing L-leucine and D-Leu-OMe-HCl to mouse skeletal muscle cells C2C12 grown in a medium without L-leucine, respectively.

FIG. 14 illustrates a comparison by measuring the number of differentiated intracellular nuclei after giving media containing L-leucine and D-Leu-OMe-HCl to mouse skeletal muscle cells C2C12 grown in a medium without L-leucine, respectively.

FIG. 15 illustrates an observation of differentiation degrees of mouse skeletal muscle cells on days 1, 6, and 9 after giving media containing L-leucine and D-Leu-BOC—H2O to mouse skeletal muscle cells C2C12 grown in a medium without L-leucine, respectively.

FIG. 16 illustrates a comparison by measuring the number of differentiated intracellular nuclei after giving media containing L-leucine and D-Leu-BOC—H2O to mouse skeletal muscle cells C2C12 grown in a medium without L-leucine, respectively.

### DETAILED DESCRIPTION

Hereinafter, example embodiments will be described in detail with reference to the accompanying drawings. However, since various changes may be made to the example embodiments, the scope of right of the patent application is not limited or restricted by these example embodiments. It should be understood that all modifications, equivalents and substitutes for the example embodiments are included within the scope of right.

The terms used in the example embodiments are used for description purposes only and should not be construed as being limited by these example embodiments. The terms in singular form may include plural forms unless otherwise specified. It will be understood that the terms “comprising” or “having,” when used herein, specify the presence of stated features, integers, steps, operations, elements, components, or combinations thereof, but do not preclude the presence or addition of one or more other features, integers, steps, operations, elements, components, or combinations thereof.

Unless otherwise defined, all technical and scientific terms used in the example embodiments have the same meanings as commonly understood by those skilled in the technical field to which the example embodiments pertain. It will be further understood that terms, such as those defined in commonly used dictionaries, should be interpreted as having a meaning that is consistent with their meanings of the context of the relevant art and the present disclosure, and will not be interpreted in an idealized or overly formal sense unless expressly so defined herein.

In addition, in the description with reference to the accompanying drawings, the same reference numerals are assigned to the same components regardless of the reference numerals, and redundant descriptions thereof will be omitted. In describing the example embodiments, when it is determined that a detailed description of related known technologies may unnecessarily obscure the gist of the example embodiments, the detailed description thereof will be omitted.

According to an example embodiment of the present disclosure, there is provided a pharmaceutical composition for preventing or treating sarcopenia containing, as an active ingredient, a non-natural amino acid derivative consisting of at least one selected from the group consisting of following Formulas 1 to 19 or a mixture of X₂ and at least one selected from the group consisting of following Formulas 1 to 19:

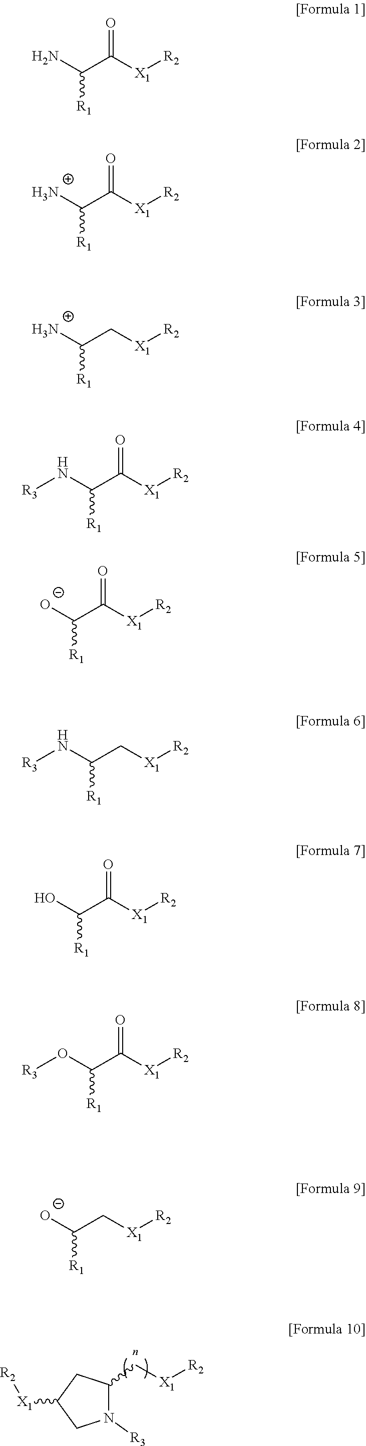

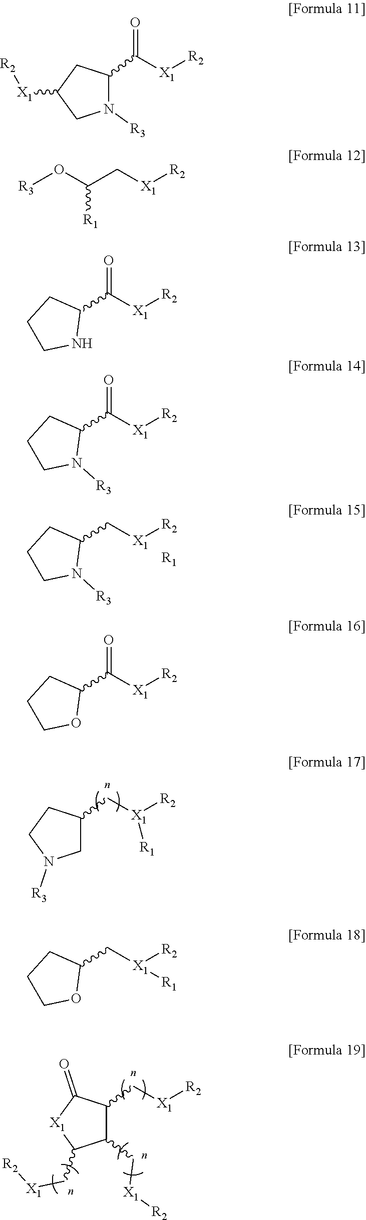

in which,

R₁ is hydrogen, a C1-C10 carboxylic acid, a C1-C10 alkyl group, C1-C10 amine, C₁-C10 alcohol, guanidine, phenyl, phenol, indole, or imidazole;

R₂ is hydrogen, a C1-C10 alkyl group, a C1-C10 aryl alkyl group, or D-glucose;

R₃ is t-butyl carbamate, benzyl carbamate, 9-fluorenylmethyl carbamate, an amine group, an ester group or acetamide;

X₁ is N, O, P, S, OH or NH₂;

X₂ is Cl⁻, Br⁻, I⁻, p-toluenesulfonate, CF₃COOH, Na⁺, Mg²⁺, K⁺, Ca²⁺, H₂O or HCl; and

n is 0, 1 or 2.

As used herein, the term “prevention” means all actions for inhibiting or delaying the onset of sarcopenia by administration of a composition. As used herein, the term “treatment” means all actions involved in alleviating or beneficially changing symptoms of sarcopenia by administration of a composition.

The non-natural amino acid derivative may be in the form represented by the following Structural Formulas 1 to 3,

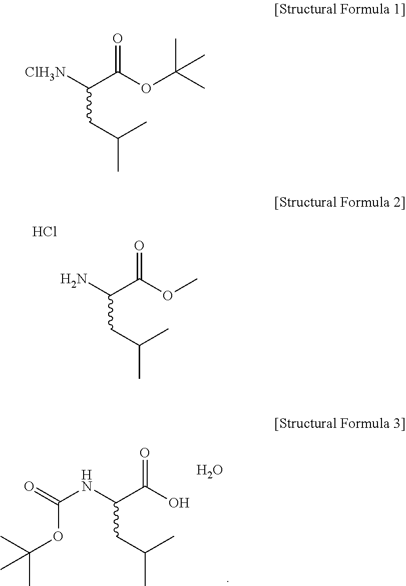

More specifically, it may be at least one selected from the group consisting of L-Leu-OtBu-HCl, D-Leu-OMe-HCl and D-Leu-BOC—H2O.

The L-Leu-OtBu-HCl may be combined with HCl in L-Leu-OtBu or correspond to a mixed form, the D-Leu-OMe-HCl may be combined with HCl in D-Leu-OMe or correspond to a mixed form, and the D-Leu-BOC—H2O may be combined with H₂O in D-Leu-BOC or correspond to a mixed form.

The pharmaceutical composition may inhibit myostatin mRNA or protein expression, and may reduce a myostatin promoter activity, so that it is possible to more fundamentally prevent and treat muscle loss and muscle strength reduction.

The pharmaceutical composition for preventing or treating sarcopenia according to an example embodiment of the present disclosure may contain the non-natural amino acid derivative at a concentration of 0.001 mM or more, specifically, it may contain the same at a concentration of 0.001 mM to 10 mM.

The pharmaceutical composition of the present disclosure may be prepared by a method known in the pharmaceutical field in order to be used as pharmaceuticals, and is mixed with a pharmaceutically acceptable carrier, an excipient, a diluent, a stabilizer, a preservative, etc. to be prepared and used in a formulation such as powder, granule, tablet, capsule or injection. In addition, the composition may be prepared as a sustained-release preparation so that the release of an active ingredient occurs slowly, including a base used for sustained-release purpose in addition to the active ingredient.

Pharmaceutically acceptable carriers may further include, for example, carriers for oral administration or carriers for parenteral administration. The carriers for oral administration may include lactose, starch, cellulose derivatives, magnesium stearate and stearic acid. In addition, it may include various drug delivery materials used for oral administration to a peptide agent. In addition, the carriers for parenteral administration may include water, suitable oils, saline, aqueous glucose and glycols, and further include stabilizers and preservatives. Suitable stabilizers include antioxidants such as sodium hydrogen sulfite, sodium sulfite or ascorbic acid. Suitable preservatives include benzalkonium chloride, methyl-or propyl-parabens and chlorobutanol. The pharmaceutical composition of the present disclosure may further include a lubricant, a wetting agent, a sweetening agent, a flavoring agent, an emulsifier, a suspending agent, etc. in addition to the above components.

Examples of the diluent include non-aqueous solvents such as propylene glycol, polyethylene glycol, and vegetable oil such as olive oil and peanut oil, and aqueous solvents such as salt water (preferably 0.8% of salt water) and water including a buffered medium (preferably 0.05 M of phosphate buffer), but are not limited thereto.

Examples of the excipient include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like, but are not limited thereto.

Examples of the stabilizer include carbohydrates such as sorbitol, mannitol, starch, sucrose, dextran, glutamate, and glucose, or proteins such as animal, vegetable, or microbial proteins such as milk powder, serum albumin, casein and the like, but are not limited thereto.

Examples of the preservative may include thimerosal, merthiolate, gentamicin, neomycin, nystatin, amphotericin B, tetracycline, penicillin, streptomycin, polymyxin B and the like, but are not limited thereto.

The pharmaceutical composition of the present disclosure may be administered to a mammal including a human using any method. For example, the pharmaceutical composition may be orally or parenterally administered. A parenteral administration method may include intravenous, intramuscular, intra-arterial, intramedullary, intradural intracardiac, percutaneous, subcutaneous, intraperitoneal, intranasal, intestinal, local, sublingual or intrarectal administration, but is not limited thereto.

The pharmaceutical composition of the present disclosure may be formulated into preparations for oral or parenteral administration, depending on the route of administration as described above.

The total effective amount of the pharmaceutical composition of the present disclosure may be administered to a patient in a single dose, and may be administered by a fractionated treatment protocol that is administered for a long period of time in multiple doses. The pharmaceutical composition of the present disclosure may vary the content of the active ingredient according to the severity of the disease. The dosage thereof may be determined in consideration of various factors not only preparation methods, routes of administration, and the number of treatments, but also the patient's age, weight, health condition, disease severity, administration time and method. In consideration thereof, those skilled in the pertinent technical field will be able to determine an appropriate effective dosage of the composition of the present disclosure. The pharmaceutical composition according to the present disclosure is not particularly limited in its formulation, route of administration, and method of administration as long as it exhibits the effects of the present disclosure.

Hereinafter, the present disclosure will be described in more detail by way of examples. However, these examples are only for illustrating the present disclosure, and the scope of the present disclosure is not limited to these examples.

### Example 1. Inhibitory Effect of Myostatin Promoter Activity

### 1) Experiment on Inhibitory Effect of Myostatin Promoter Activity by L-Leu-OtBu-HCl

After seeding 1×10⁵ cells of mouse skeletal muscle cells C2C12 (ATCC, US) in a 12-well culture plate, on the next day, pGL4.15 empty vector and pGL4.15-MSTN vector containing myostatin promoter were transfected using Lipofectamine 2000 for 4 hours. After 4 hours, the complete medium (0.8 mM L-leucine) and the medium without L-leucine were treated with L-Leu-OtBu-HCl at a concentration of 0.1 mM. After one day, a dual luciferase assay kit (Promega Inc.) was used to measure the promoter activity of myostatin.

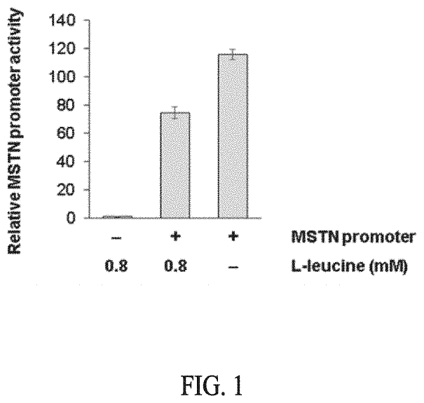

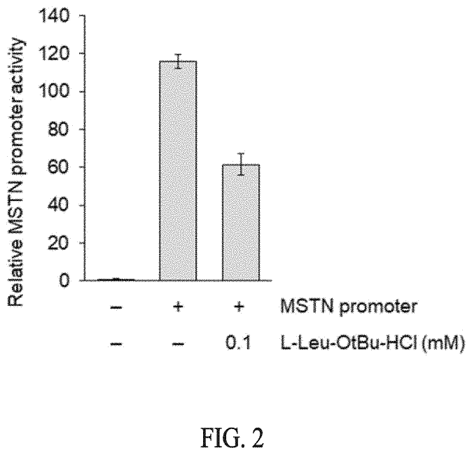

As a result, it was identified that the myostatin promoter activity of C2C12 cells in the medium without L-leucine was increased by 55% compared to the complete medium (see FIG. 1), and that the myostatin promoter activity was significantly reduced when treated with L-Leu-OtBu-HCl at a concentration of 0.1 mM in the medium without L-leucine (see FIG. 2).

### 2) Experiment and Comparison of Inhibitory Effect of Myostatin Promoter Activity by L-leucine and D-Leu-OMe-HCl

In the same manner as the experiment to examine the inhibitory effect of a myostatin promoter activity by L-Leu-OtBu-HCl, L-leucine was treated at concentrations of 0.1 mM and 1 mM, and D-Leu-OMe-HCl was treated at concentrations of 0.1 mM and 1 mM, respectively, in a medium without L-leucine, and a dual luciferase assay kit (Promega Inc.) was used to measure the promoter activity of myostatin.

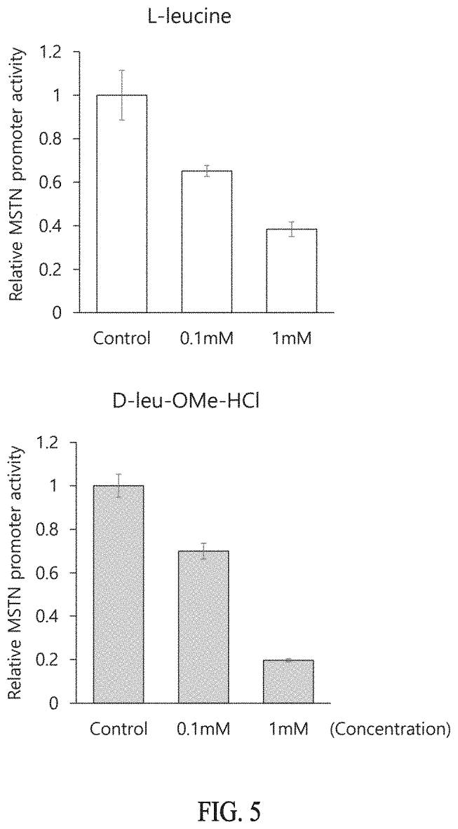

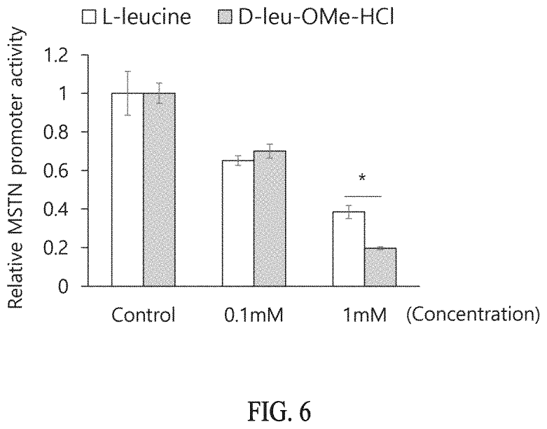

As a result, it was observed that both L-leucine and D-Leu-OMe-HCl inhibited the myostatin promoter activity of mouse skeletal muscle cells C2C12, and it was identified that D-Leu-OMe-HCl showed a relatively large decrease in myostatin promoter activity at a concentration of 1 mM (about 1.96 times) compared to L-leucine (see FIGS. 5 and 6).

### 3) Experiment and Comparison of Inhibitory Effect of Myostatin Promoter Activity by L-leucine and D-Leu-BOC—H2O

In the same manner as in the experiment to examine the inhibitory effect of a myostatin promoter activity by L-Leu-OtBu-HCl, L-leucine was treated at concentrations of 0.1 mM and 1 mM, and D-Leu-BOC—H2O was treated at concentrations of 0.1 mM and 1 mM, respectively in a medium without L-leucine, and a dual luciferase assay kit (Promega Inc.) was used to measure the promoter activity of myostatin.

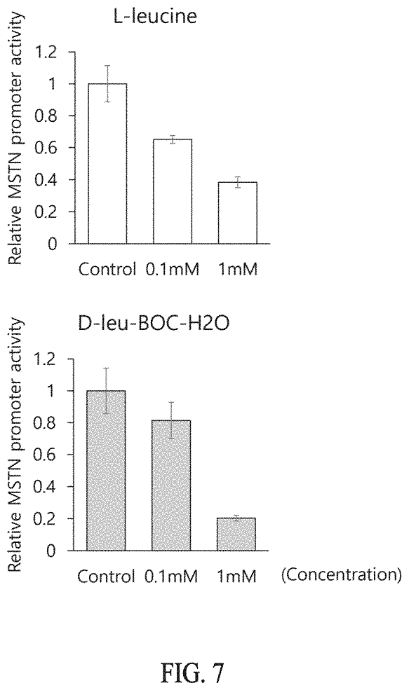

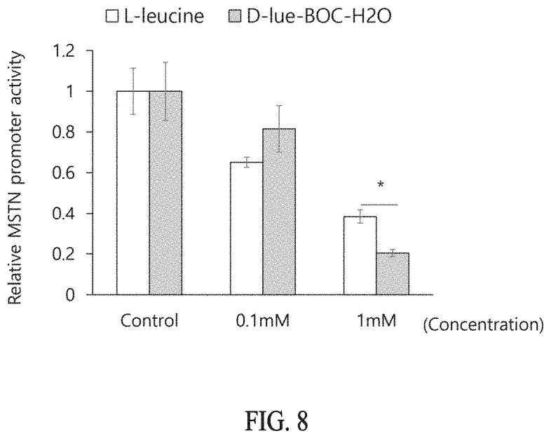

As a result, it was observed that both L-leucine and D-Leu-BOC—H2O inhibited the myostatin promoter activity of mouse skeletal muscle cells C2C12, and it was identified that D-Leu-BOC—H2O showed a relatively large decrease in myostatin promoter activity at a concentration of 1 mM (about 1.89 times) compared to L-leucine (see FIGS. 7 and 8).

### Example 2. Inhibitory Effect of Myostatin mRNA Expression

### 1) Experiment of Inhibitory Effect of Myostatin mRNA Expression by L-Leu-OtBu-HCl

After seeding mouse skeletal muscle cells C2C12 (ATCC, US) 3×10⁵ cells in a 6-well culture plate, on the next day, L-Leu-OtBu-HCl was treated at a concentration of 0.1 mM in a complete medium (0.8 mM L-leucine) and a medium without L-leucine. After one day, the mRNA expression level of myostatin was identified using qRT-PCR, and the myostatin mRNA expression value was corrected using the expression value of beta-actin.

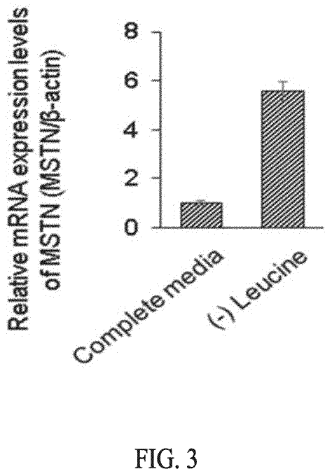

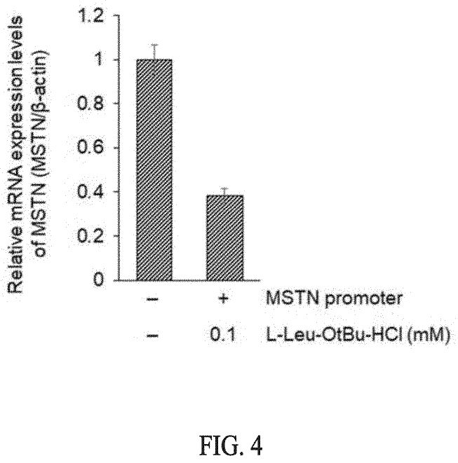

As a result, it was observed that the myostatin mRNA expression of C2C12 cells in the medium without L-leucine was increased 5.5-fold compared to the complete medium containing L-leucine at a concentration of 0.8 mM (see FIG. 3), and in the medium without L-leucine, when L-Leu-OtBu-HCl at a concentration of 0.1 mM was treated, the expression of myostatin mRNA was decreased (see FIG. 4).

### 2) Experiment and Comparison of Inhibitory Effect of Myostatin mRNA Expression by L-leucine and D-Leu-OMe-HCl

In the same manner as the experiment to examine the inhibitory effect of myostatin mRNA expression by L-Leu-OtBu-HCl, L-leucine was treated at concentrations of 0.1 mM and 1 mM, and D-Leu-OMe-HCl was treated at concentrations of 0.1 mM and 1 mM, respectively, in a medium without L-leucine, the myostatin mRNA expression level was identified using qRT-PCR, and the myostatin mRNA expression value was corrected using the expression value of beta-actin.

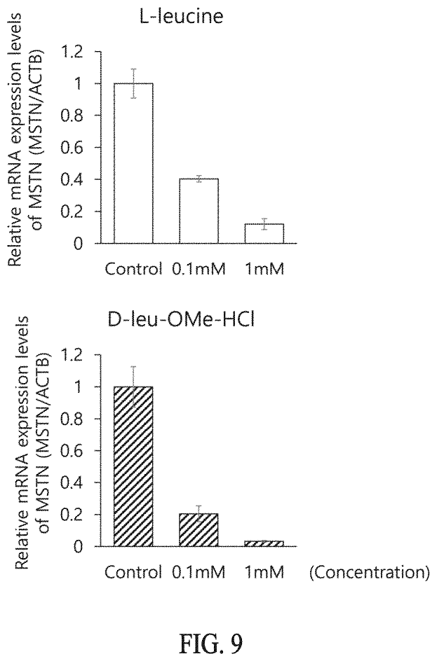

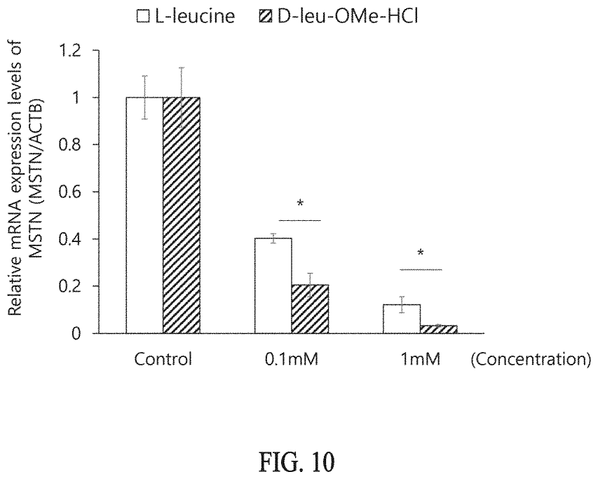

As a result, it was observed that both L-leucine and D-Leu-OMe-HCl inhibited the myostatin mRNA expression of mouse skeletal muscle cells C2C12, and it was identified that D-Leu-OMe-HCl showed a relatively large decrease in myostatin mRNA expression level at concentrations of 0.1 mM and 1 mM (about 1.97 times and about 3.68 times, respectively) compared to L-leucine (see FIGS. 9 and 10).

### 3) Experiment and Comparison of Inhibitory Effect of Myostatin mRNA Expression by L-leucine and D-Leu-BOC—H2O

In the same manner as the experiment to examine the inhibitory effect of myostatin mRNA expression by L-Leu-OtBu-HCl, L-leucine was treated at concentrations of 0.1 mM and 1 mM, and D-Leu-BOC—H2O was treated at concentrations of 0.1 mM and 1 mM, respectively, in a medium without L-leucine, the myostatin mRNA expression level was identified using qRT-PCR, and the myostatin mRNA expression value was corrected using the expression value of beta-actin.

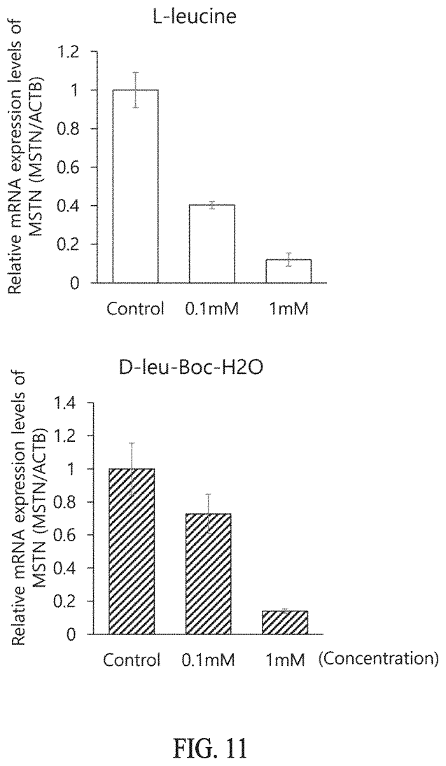

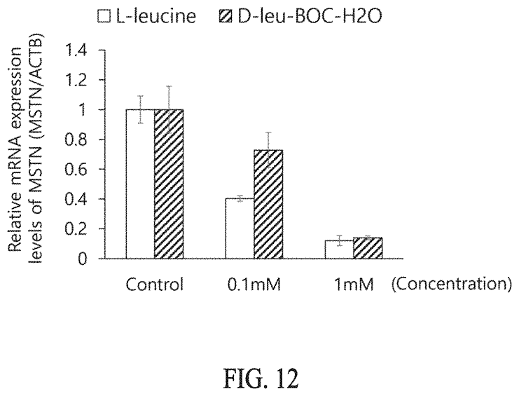

As a result, it was observed that both L-leucine and D-Leu-BOC—H2O inhibited the myostatin mRNA expression of mouse skeletal muscle cells C2C12, and it was identified that D-Leu-BOC—H2O decreased the expression level of myostatin mRNA at a concentration of 1 mM similarly to L-leucine (see FIGS. 11 and 12).

### Example 3. Measurement of Degree of Differentiation of Mouse Skeletal Muscle Cells C2C12 and Number of Differentiated Intracellular Nuclei

### 1) Cell Differentiation Analysis Experiment and Comparison by L-Leucine and D-Leu-OMe-HCl

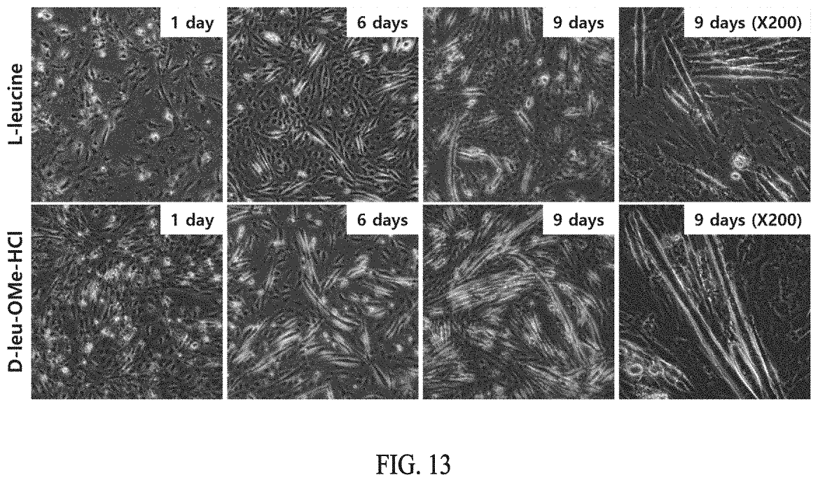

To observe how L-leucine and D-Leu-OMe-HCl act on the differentiation of mouse skeletal muscle cells C2C12, a differentiation medium containing 1 mM L-leucine and D-Leu-OMe-HCl was given to mouse skeletal muscle cells C2C12 continuously grown in a medium without L-leucine, respectively. On days 1, 6, and 9, the degree of differentiation of mouse skeletal muscle cells and the number of nuclei present in the differentiated cells were measured (see FIG. 13).

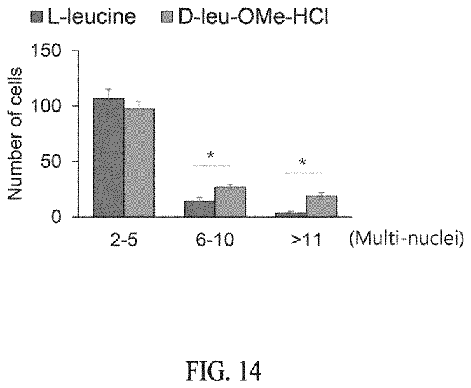

As a result, compared to L-leucine, a relatively large number of cells in which differentiation occurs were observed in the cells to which D-Leu-OMe-HCl was added, and cells containing 6 or more nuclei were observed statistically significantly in cells to which D-Leu-OMe-Hcl was added (see FIG. 14).

### 2) Cell Differentiation Analysis Experiment and Comparison by L-Leucine and D-Leu-BOC—H2O

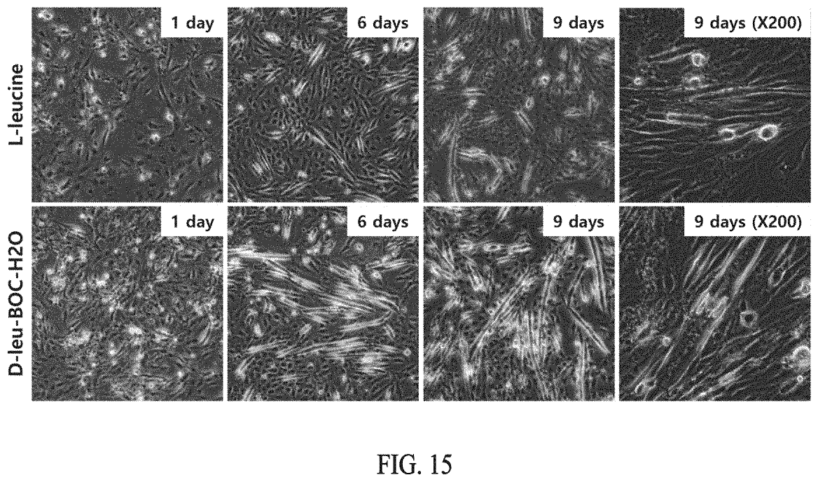

To observe how L-leucine and D-Leu-BOC—H2O act on the differentiation of mouse skeletal muscle cells C2C12, a differentiation medium containing 1 mM L-leucine and D-Leu-BOC—H2O was given to mouse skeletal muscle cells C2C12 continuously grown in a medium without L-leucine, respectively. On days 1, 6, and 9, the degree of differentiation of mouse skeletal muscle cells and the number of nuclei present in the differentiated cells were measured (see FIG. 15).

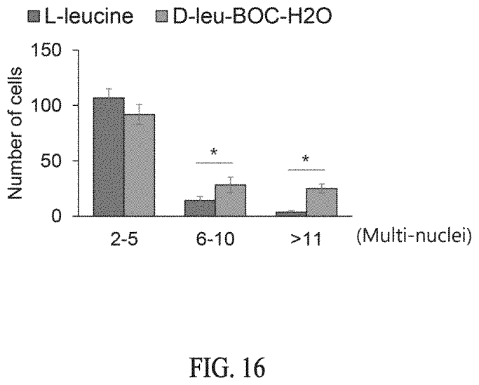

As a result, compared to L-leucine, a relatively large number of cells in which differentiation occurs were observed in the cells to which D-Leu-BOC—H2O was added, and cells containing 6 or more nuclei were observed statistically significantly in cells to which D-Leu-BOC—H2O was added (see FIG. 16).

The results of the above examples suggest that the non-natural amino acid derivative of the present disclosure can be usefully used for the prevention or treatment of sarcopenia by inhibiting myostatin mRNA expression and promoter activity in muscle cells.

Although the example embodiments have been described based on the limited drawings as described above, those skilled in the pertinent technical field may apply various technical modifications and variations based thereon. For example, even when the described technologies are performed in a different order from that in the described method, and/or the described components are coupled or combined in a manner different from that as described above, or are replaced or substituted with other components or equivalents, appropriate results may be achieved.

Therefore, other implementations, other example embodiments, and equivalents to claims also fall within the scope of the following claims.

1. A method for preventing or treating sarcopenia, the method comprising:
administering to a subject a composition containing as an active ingredient, a non-natural amino acid derivative consisting of at least one selected from the group consisting of following Formulas 1 to 19 or a mixture of X₂ and the at least one selected from the group consisting of following Formulas 1 to 19: 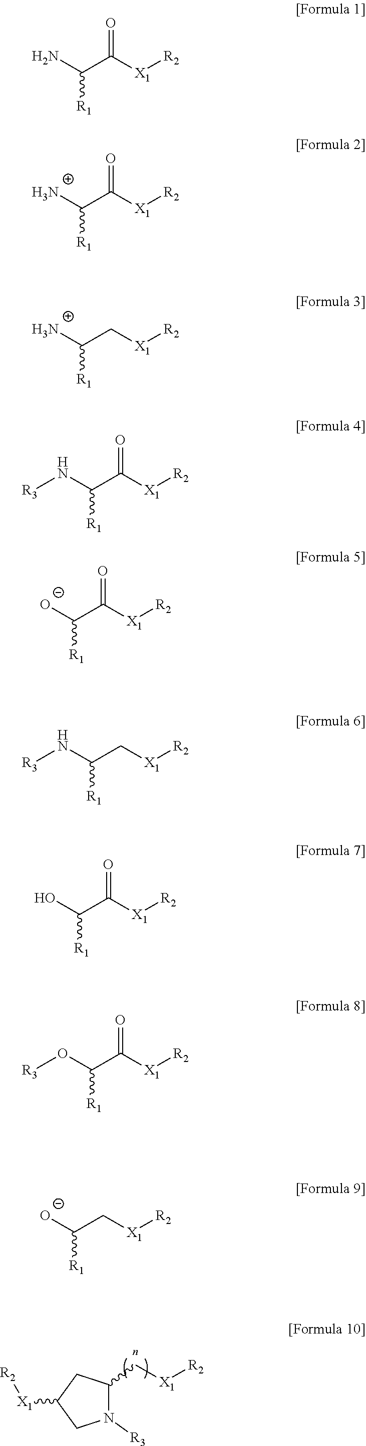 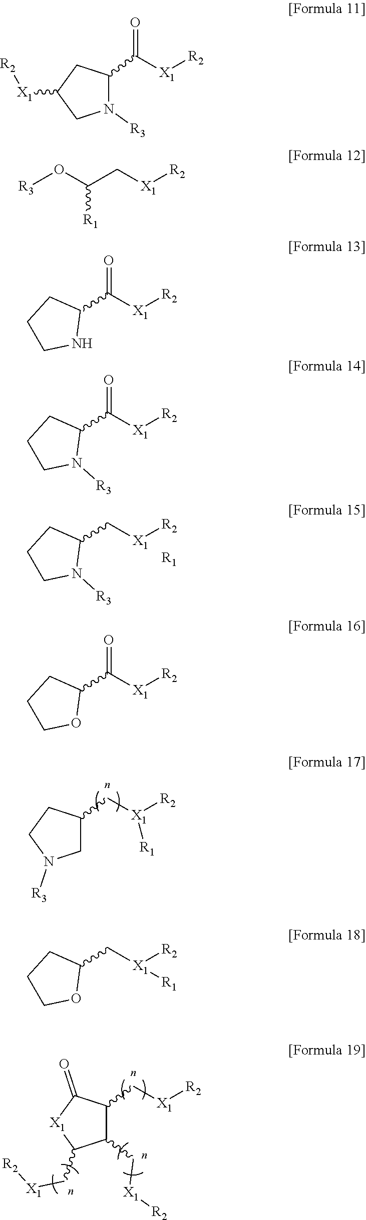
wherein,
R₁ is hydrogen, a C1-C10 carboxylic acid, a C1-C10 alkyl group, C1-C10 amine, C1-C10 alcohol, guanidine, phenyl, phenol, indole, or imidazole;
R₂ is hydrogen, a C1-C10 alkyl group, a C1-C10 aryl alkyl group, or D-glucose;
R₃ is t-butyl carbamate, benzyl carbamate, 9-fluorenylmethyl carbamate, an amine group, an ester group or acetamide;
X₁ is N, O, P, S, OH or NH₂;
X₂ is Cl⁻, Br⁻, I⁻, p-toluenesulfonate, CF₃COOH, Na⁺, Mg²⁺, K⁺, Ca²⁺, H₂O or HCl; and
n is 0, 1 or 2.

2. The method of claim 1, wherein the composition inhibits myostatin mRNA or protein expression.

3. The method of claim 1, wherein the composition reduces a myostatin promoter activity.

4. The method of claim 1, wherein the non-natural amino acid derivative is at least one selected from the group consisting of D-Leu-OtBu-HCl, D-Leu-OMe-HCl and D-Leu-BOC—H2O.

5. The method of claim 1, wherein the composition contains 0.001 mM to 10 mM of the non-natural amino acid.

6. A preparation for preventing or treating sarcopenia comprising at least one selected from the group consisting of a pharmaceutically acceptable carrier, an excipient, a diluent, a stabilizer and a preservative, and the composition of claim 1.

7. The preparation of claim 6, wherein the pharmaceutically acceptable carrier includes carriers for oral administration or carriers for parenteral administration.

8. The preparation of claim 6, wherein the excipient includes at least one selected from the group consisting of starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol.

9. The preparation of claim 6, wherein the diluent includes at least one selected from the group consisting of propylene glycol, polyethylene glycol, olive oil, peanut oil, salt water, water, and phosphate buffer.

10. The preparation of claim 6, wherein the preservative includes at least one selected from the group consisting of thimerosal, merthiolate, gentamicin, neomycin, nystatin, amphotericin B, tetracycline, penicillin, streptomycin, and polymyxin B.

11. The preparation of claim 6, wherein the preparation has a formulation of powder, granule, tablet, capsule or injection.

12. The method of claim 1, the subject is a subject whose muscle strength is weakened and/or muscle loss has progressed, or a subject with a genetic predisposition to sarcopenia.

13. The method of claim 1, the method further comprising measuring an expression level of myostatin in muscle cells of the subject before and after the administering.
